(19) United States
(12) Patent Application Publication
(10) Pub. No.: US 2011/0117212 A1
(43) Pub. Date: May 19, 2011

(54) THERAPEUTIC COMBINATION COMPRISING AN AURORA KINASE INHIBITOR AND AN ANTINEOPLASTIC AGENT

(75) Inventors: Enrico Pesenti, Parabiago (MI) (IT); Maurizio D'Incalci, Milan (IT); Dario Ballinari, San Donato Milanese (MI) (IT); Juergen Moll, Appiano Gentile (CO) (IT)

(73) Assignee: NERVIANO MEDICAL SCIENCES S.R.L., Nerviano (IT)

(21) Appl. No.: 13/054,646

(22) PCT Filed: Jul. 3, 2009
(86) PCT No.: PCT/EP2009/058413

§ 371 Date: Jan. 18, 2011

Related U.S. Application Data
(60) Provisional application No. 61/083,232, filed on Jul. 24, 2008.

(51) Int. Cl. A61K 33/24; A61K 31/496; A61K 31/7068; A61P 35/00; A61P 43/00

(52) U.S. Cl. 424/649; 514/254.06; 514/49

(57) ABSTRACT

The present invention provides a therapeutic combination comprising (a) compound 1 of formula (A) as set forth in the specification and (b) one or more antineoplastic agents selected from the group consisting of platinum derivatives, antimetabolite agents, topoisomerase I inhibitors and antimicrotubule agents, wherein the active ingredients are present in each case in free form or in the form of a pharmaceutically acceptable salt or any hydrate thereof.

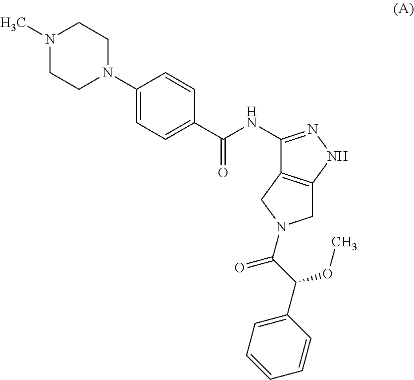

### FIELD OF THE INVENTION

The present invention relates in general to the field of cancer treatment and, more particularly, provides an anti-tumor composition comprising an aurora kinase inhibitor and a platinum derivative and/or an antimetabolite agent and/or a topoisomerase I inhibitor and/or an antimicrotubule agent having a synergistic or additive antineoplastic effect.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis, glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology, 1999, 3, 459-465.

Among the several protein kinases known in the art as being implicated in the growth of cancer cells are Aurora kinases, in particular Aurora-2.

Aurora-2 was found to be over-expressed in a number of different tumour types. Its gene locus maps at 20q13, a chromosomal region frequently amplified in many cancers, including breast [Cancer Res. 1999, 59(9), 2041-4] and colon cancers.

20q13 amplification correlates with poor prognosis in patients with node-negative breast cancer and increased Aurora-2 expression is indicative of poor prognosis and decreased survival time in bladder cancer patients [J. Natl. Cancer Inst., 2002, 94(17), 1320-9]. For a general reference to the Aurora-2 role in the abnormal centrosome function in cancer see also Molecular Cancer Therapeutics, 2003, 2, 589-595.

Several heterocyclic compounds are known in the art as protein kinase inhibitors. Among them, 3-carboxamido-pyrazoles and 3-ureido-pyrazoles and derivatives thereof, have been disclosed as protein kinase inhibitors in the international patent applications WO01/12189, WO12188, WO02/48114 and WO02/70515.

Fused bicyclic compounds comprising pyrazole moieties and possessing kinase inhibitory activity have been also disclosed in WO00/69846, WO02/12242, WO03/028720 and WO03/97610.

Specific tetrahydropyrrolo[3,4-c]pyrazole derivatives have been revealed to be potent ATP-competitive inhibitors of Aurora kinases (Fancelli, D., et al.: Journal of Medicinal Chemistry. 2005, vol. 48, no. 8, p. 3080-3084. PCT/WO 2005005427).

Surprisingly, it has been found that the antineoplastic effect, i.e. especially the delay of progression or treatment of a proliferative disease, in particular the treatment of a tumour that is refractory to other chemotherapeutics known as antineoplastic agents, of the tetrahydropyrrolo[3,4-c]pyrazole derivatives above is greatly enhanced when they are administered in combination with certain antineoplastic agents. In particular, the antineoplastic effect of such combination is greater than the effects that can be achieved with either type of combination component alone, i.e. greater than the effects of a monotherapy using only one of the combination components.

The present invention thus provides new combinations of a tetrahydropyrrolo[3,4-c]pyrazole derivative with known pharmaceutical agents that are particularly suitable for the treatment of proliferative disorders, especially cancer. More specifically, the combinations of the present invention are very useful in therapy as antitumor agents and lack, in terms of both toxicity and side effects, the drawbacks associated with currently available antitumor drugs.

### SUMMARY OF THE INVENTION

The present invention provides, in a first aspect, a therapeutic combination comprising (a) Compound 1 of formula (A):

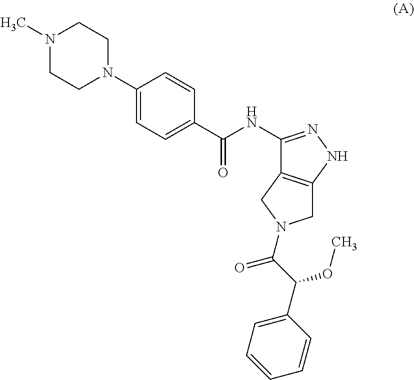

and (b) one or more antineoplastic agents selected from the group consisting of platinum derivatives, antimetabolite agents, topoisomerase I inhibitors and antimicrotubule agents, wherein the active ingredients are present in each case in free form or in the form of a pharmaceutically acceptable salt or any hydrate thereof.

The present invention also provides a combined preparation for simultaneous, separate or sequential use of the combination as described above.

The present invention also provides a pharmaceutical composition comprising a combination as described above which is further admixed with a pharmaceutically acceptable carrier, diluent or excipient.

The present invention further provides a method of treating or delaying the progression of a proliferative disorder by administering to a patient in need thereof a therapeutically effective amount of the combination as described above.

### DETAILED DESCRIPTION OF THE INVENTION

The present invention provides, in a first embodiment, a therapeutic combination comprising (a) Compound 1 of formula (A):

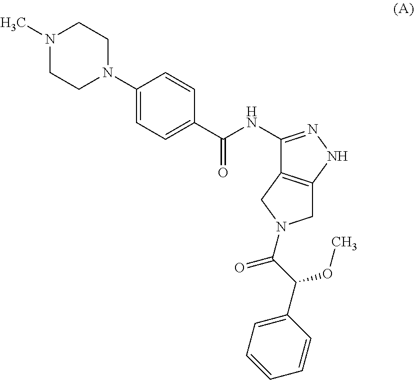

and (b) one or more antineoplastic agents selected from the group consisting of platinum derivatives, antimetabolite agents, topoisomerase I inhibitors and antimicrotubule agents, wherein the active ingredients are present in each case in free form or in the form of a pharmaceutically acceptable salt or any hydrate thereof.

In a further embodiment the combination according to the invention is a combined preparation for simultaneous, separate or sequential use.

A further embodiment relates to the combination according to the invention in a method of treating or delaying the progression of a proliferative disorder, wherein the method comprises the simultaneous, sequential or separate administration to a subject in need thereof of the therapeutic combination.

In a still further embodiment the invention provides a pharmaceutical composition comprising a therapeutic combination admixed with a pharmaceutically acceptable carrier, diluent or excipient.

Another embodiment relates to the use of compound 1 of formula (A) as defined above in the preparation of a medicament for the treatment of a proliferative disorder, wherein said treatment comprises simultaneously, sequentially or separately administering compound 1 of formula (A) as defined above and one or more antineoplastic agents selected from the group consisting of platinum derivatives, antimetabolite agents, topoisomerase I inhibitors and antimicrotubules agents, to a subject.

The compound 1 of formula (A) has the chemical name N-{5-[(2R)-2-methoxy-2-phenylethanoyl]-1,4,5,6-tetrahydropyrrolo[3,4-c]pyrazol-3-yl}-4-(4-methylpiperazin-1-yl)benzamide. It can be prepared as described in WO2005/005427 (incorporated herein by reference), is endowed with protein kinase inhibitory activity and is thus useful in therapy as antitumor agent.

Pharmaceutically acceptable salts of compound 1 of formula (A) include the acid addition salts with inorganic or organic acids, e.g., nitric, hydrochloric, hydrobromic, sulphuric, perchloric, phosphoric, acetic, trifluoroacetic, propionic, glycolic, lactic, oxalic, malonic, malic, maleic, mesylate, tartaric, citric, benzoic, cinnamic, mandelic, methanesulphonic, isethionic and salicylic acid and the like.

According to a preferred embodiment of the invention, the platinum derivative is selected from the group consisting of cisplatin, oxaliplatin, carboplatin, nedaplatin, lobaplatin and satraplatin. Oxaliplatin can be administered, e.g., in the form as it is marketed, e.g. under the trademark ELOXATIN®.

According to a more preferred embodiment of the invention the platinum derivative is cisplatin or oxaliplatin.

An antimetabolite agent includes, but is not limited to 5-fluorouracil (5-FU), capecitabine, gemcitabine, cytosine arabinoside (Ara-C), pemetrexed, methotrexate and edatrexate. Capecitabine can be administered, e.g., in the form as it is marketed, e.g. under the trademark XELODA®. Gemcitabine can be administered, e.g., in the form as it is marketed, e.g. under the trademark GEMZAR®. Pemetrexed can be administered, e.g., in the form as it is marketed, e.g. under the trademark ALIMTA®.

According to a more preferred embodiment of the invention the antimetabolite agent is gemcitabine, 5-FU or Ara-C.

A topoisomerase I inhibitor includes, but is not limited to topotecan, irinotecan, SN-38 and 9-nitrocamptothecin. Irinotecan can be administered, e.g., in the form as it is marketed, e.g. under the trademark CAMPTOSAR®. Topotecan can be administered, e.g., in the form as it is marketed, e.g. under the trademark HYCAMTIN SN-38 is the active metabolite of irinotecan, obtained by in vivo hydrolysis of irinotecan.

According to a more preferred embodiment of the invention the topoisomerase I inhibitor is Irinotecan or SN38.

An antimicrotubule agent includes, but is not limited to paclitaxel, docetaxel and epothilons derivatives. Paclitaxel can be administered, e.g., in the form as it is marketed, e.g. under the trademark TAXOL®. Docetaxel can be administered, e.g., in the form as it is marketed, e.g. under the trademark TAXOTERE®.

According to a more preferred embodiment of the invention the antimicrotubule agent is docetaxel and paclitaxel.

In the present invention, each of the active ingredients of the combination is provided in an amount effective to produce a synergistic antineoplastic effect.

The present invention also provides a method for lowering the side effects caused by antineoplastic therapy with an antineoplastic agent in mammals, including humans, in need thereof, the method comprising administering to said mammal a combined preparation comprising compound 1 of formula (A) as defined above and one or more antineoplastic agents selected from the group consisting of platinum derivatives, antimetabolite agents, topoisomerase I inhibitors and antimicrotubule agents, in amounts effective to produce a synergistic antineoplastic effect.

By the term “a synergistic antineoplastic effect” as used herein is meant the inhibition of the growth of the tumor, preferably the complete regression of the tumor, by administering an effective amount of the combination of compound 1 of formula (A) as defined above and a platinum derivative, an antimetabolite agent, a topoisomerase I inhibitor to mammals or an antimicrotubule agent to mammals, including human.

The term “combined preparation” as used herein defines especially a “kit of parts” in the sense that the combination components (a) and (b) as defined above can be dosed independently or by use of different fixed combinations with distinguished amounts of the combination components (a) and (b), i.e. simultaneously or at different time points. The elements of the kit of parts can then, e.g., be administered simultaneously or chronologically staggered, that is at different time points and with equal or different time intervals for any element of the kit of parts. Most preferably, the time intervals are chosen such that the effect on the treated disease in the combined use of the parts is greater than the effect which would be obtained by use of only any one of the combination components (a) and (b). The ratio of the total amounts of the combination component (a) to the combination component (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, less toxicity, or a combined therapeutic effect in a non-effective dosage of one or both of the combination components (a) and (b), and very preferably a strong synergism of the combination components (a) and (b).

By the term “administered” or “administering” as used herein is meant parenteral and/or oral administration. By “parenteral” is meant intravenous, subcutaneous and intramuscular administration.

In the method of the subject invention, for the administration of the compound of formula (A), the course of therapy generally employed is in the range from 100 mg/m²/day to 1500 mg/m²/day of body surface area for up to 21 consecutive days. More preferably, the course therapy employed is from about 150 mg/m²/day to about 350 mg/m²/day of body surface area for up to 21 consecutive days.

In a particularly preferred regimen, the compound of formula (A) is administered in a dose of 190 or 250 mg/m²/day of body surface area for three hours infusion on day 1 and 8 of a three weeks cycle. Other possible therapeutic schedules are disclosed, for example, in WO2008/052931 published May 8, 2008 (incorporated herein by reference).

The compound of formula (A) can be administered in a variety of dosage forms, e.g., orally, in the form of tablets, capsules, sugar or film coated tablets, liquid solutions or suspensions; rectally in the form of suppositories; parenterally, e.g., intramuscularly, or through intravenous and/or intrathecal and/or intraspinal injection or infusion.

In the method of the subject invention, for the administration of a platinum derivative, preferably carboplatin, the course of therapy generally employed depends on the systemic exposure (expressed as AUC value), the renal function of the patient and on the schedule of administration. A regimen targeting an AUC of from 2 to 6 over a 1 to 4 week schedule is usually adopted. More preferably, a regimen targeting an AUC of from 4 to 6 over a 4-week schedule is used. For the administration of oxaliplatin, the course of therapy generally employed is from 10 mg/m²/day to 100 mg/m²/day every 2-3 weeks. More preferably, the course of therapy generally employed is from about 30 mg/m²/day to 85 mg/m²/day on day 1, once every 2 weeks.

For the administration of an antimetabolite agent, preferably gemcitabine, the course of therapy generally employed is from 200 mg/m²/day to 2000 mg/m²/day as weekly administration. More preferably, the course of therapy generally employed is from about 800 mg/m²/day to 1250 mg/m²/day on days 1 and 8 of a 21-day cycle or on days 1, 8 and 15 of a 28-day cycle.

For the administration of a topoisomerase I inhibitor, preferably topotecan, the course of therapy generally employed is from 0.1 mg/m² to 2 mg/m² daily for 2-10 consecutive days. More preferably, the course of therapy generally employed is from about 0.5 mg/m² to 1.5 mg/m² daily for 3-5 consecutive days in a 21-day cycle.

For the administration of irinotecan, the course of therapy generally employed is from 50 mg/m² to 350 mg/m² daily on days 1, 8, 15 and 22 of a 6-week cycle. More preferably, the course of therapy generally employed is from about 125 mg/m² to 180 mg/m² daily on days 1, 8, 15 and 22 of a 6-week cycle.

For the administration of an antimicrotubules agent, preferably docetaxel, the course of therapy generally employed is from about 50 mg/m²/day to 100 mg/m²/day every three weeks or from 20 mg/m²/day to 100 mg/m²/day weekly. Preferably, administration occurs over 3 hours infusion on days 1 and 8 of a three-week cycle.

The antineoplastic therapy of the present invention is in particular suitable for treating all forms of cancer including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; haematopoietic tumors of lymphoid lineage including leukaemia, acute lymphocytic leukaemia, acute lymphoblastic leukaemia, B-cell lymphoma, T-cell-lymphoma, Hodgkin's lymphoma, non-Hodgkin's lymphoma, mantle cell lymphoma, hairy cell lymphoma and Burkett's lymphoma; haematopoietic tumors of myeloid lineage, including acute and chronic myelogenous leukemias, myelodysplastic syndrome and promyelocytic leukaemia; multiple myeloma; tumors of mesenchymal origin, including fibrosarcoma and rhabdomyosarcoma; tumors of the central and peripheral nervous system, including astrocytoma neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

As stated above, the effect of the combination of the invention is significantly increased without a parallel increased toxicity. In other words, the combined therapy of the present invention enhances the antitumoral effects of the component (a) and/or of component (b) of the combination of the invention and thus yields the most effective and less toxic treatment for tumors.

Pharmaceutical compositions according to the invention are useful in anticancer therapy.

The present invention further provides a commercial package comprising, in a suitable container means, (a) a compound of formula (A) as defined above, and (b) one or more antineoplastic agent selected from the group consisting of platinum derivatives, antimetabolite agents, topoisomerase I inhibitors, and antimicrotubule agents wherein the active ingredients are present in each case in free form or in the form of a pharmaceutically acceptable salt or any hydrate thereof, together with instructions for simultaneous, separate or sequential use thereof.

In a package according to the invention each of partner (a) and (b) are present within a single container means or within distinct container means.

Another embodiment of the present invention is a commercial package comprising a pharmaceutical composition or product as described above.

Due to the key role of the aurora kinase proteins in the regulation of cellular mitosis and proliferation, the combinations of the present invention are also useful in the treatment of a variety of cell proliferative disorders such as, for example, benign prostate hyperplasia, familial adenomatosis, polyposis, neurofibromatosis, psoriasis, vascular smooth cell proliferation associated with atherosclerosis, pulmonary fibrosis, arthritis, glomerulonephritis and post-surgical stenosis and restenosis.

The combinations of this invention, as modulators of apoptosis, may also be useful in the treatment of cancer, viral infections, prevention of AIDS development in HIV-infected individuals, autoimmune diseases and neurodegenerative disorders.

The activities of the combination of the present invention are shown for instance by the following in vitro and in vivo tests, which are intended to illustrate but not to limit the present invention.

### Example 1

### Materials and Methods for In Vitro Cytotoxic Activity Testing

Exponentially growing human ovarian carcinoma (A2780) and/or human colon carcinoma (HCT-116) cell lines were seeded and incubated at 37° C. in a humidified 5% CO₂ atmosphere. Drugs were added to the experimental culture, and incubations were carried out at 37° C. for 72 hours in the dark. Scalar doses of Compound 1 of formula (A) as defined above and antineoplastic agent were added to the medium 24 hours after seeding. One or both of the following treatment schedules were tested: simultaneous administration (both drugs administered to cells for 72 hours) and sequential administration (Compound 1 administered 24 hours after the other agent). Drug solutions were prepared immediately before use. At the end of treatment, cell proliferation was determined by an intracellular adenosine triphosphate monitoring system (CellTiterGlo-Promega) using the Envision (PerkinElmer) as reader. Inhibitory activity was evaluated comparing treated versus control data using Assay Explorer (MDL) program. The dose inhibiting 50% of cell growth was calculated using sigmoidal interpolation curve. Combination indices (C.I.) were calculated using a computer program for multiple drug effect analysis based on the equation of Chou-Talalay (Adv Enzyme Regul 1984; 22:27-55) for mutually nonexclusive drugs, where a C.I.<1 indicates a more than additive effect (C.I.>3 indicates strong antagonism; 1.3<C.I.<3, antagonism; 0.8<C.I.<1.3, additivity; 0.3<C.I.<0.8, synergism; C.I.<0.3, strong synergism).

### Example 2

### In Vitro Cytotoxic Activity of the Combination with Cisplatin

The results shown in Table 1 indicate that on both human ovarian carcinoma A2780 and human colon carcinoma HCT-116 cell lines the administration of Compound 1 in combination with cisplatin resulted in a synergistic antitumor effect.

TABLE 1
In vitro evaluation of the cytotoxic effect of Compound 1 in
combination with cisplatin

| | Drug | | | | |
|---|---|---|---|---|---|
| | Compound 1 | cisplatin | | | Effect of |
| Cell line | μM | μM | Schedule | C.I. | combination |
| A2780 | 0.5 | 10,000 | Simultaneous | 0.69 | Synergism |
| | 0.25 | 10,000 | | 0.73 | Synergism |
| | 0.5 | 10,000 | Sequential | 0.63 | Synergism |
| | 0.25 | 10,000 | | 0.74 | Synergism |
| HCT-116 | 0.5 | 10,000 | Sequential | 0.73 | Synergism |

### Example 3

### In Vitro Cytotoxic Activity of the Combination with Oxaliplatin

The results shown in Table 2 indicate that on human colon carcinoma HCT-116 cell line the administration of Compound 1 in combination with oxaliplatin resulted in a synergistic antitumor effect.

TABLE 2
In vitro evaluation of the cytotoxic effect of Compound 1 in
combination with oxaliplatin

| | Drug | | | | |
|---|---|---|---|---|---|
| | Compound 1 | oxaliplatin | | | Effect of |
| Cell line | μM | μM | Schedule | C.I. | combination |
| HCT-116 | 0.5 | 20,000 | Sequential | 0.58 | Synergism |
| | 0.25 | 20,000 | | 0.70 | Synergism |
| | 0.12 | 20,000 | | 0.79 | Synergism |

### Example 4

### In Vitro Cytotoxic Activity of the Combination with 5-FU

The results shown in Table 3 indicate that on human colon carcinoma HCT-116 cell line the administration of Compound 1 in combination with 5-FU resulted in a synergistic antitumor effect.

TABLE 3
In vitro evaluation of the cytotoxic effect of Compound 1 in
combination with 5-FU

| | Drug | | | | |
|---|---|---|---|---|---|
| | Compound 1 | 5-FU | | | Effect of |
| Cell line | μM | nM | Schedule | C.I. | combination |
| HCT-116 | 0.5 | 12.5 | Sequential | 0.77 | Synergism |
| | 0.25 | 3.1 | | 0.76 | Synergism |

### Example 5

### In Vitro Cytotoxic Activity of the Combination with SN38

The results shown in Table 4 indicate that on human ovarian carcinoma A2780 the administration of Compound 1 in combination with the topoisomerase I inhibitor SN38 resulted in a synergistic antitumor effect.

TABLE 4
In vitro evaluation of the cytotoxic effect of Compound 1 in
combination with SN38

| | Drug | | | | |
|---|---|---|---|---|---|
| | Compound 1 | SN38 | | | Effect of |
| Cell line | μM | μM | Schedule | C.I. | combination |
| A2780 | 0.5 | 0.012 | Simultaneous | 0.78 | Synergism |
| | 0.25 | 0.012 | | 0.80 | Synergism |
| | 0.5 | 0.012 | Sequential | 0.58 | Synergism |
| | 0.25 | 0.012 | | 0.56 | Synergism |

### Example 6

### In Vitro Cytotoxic Activity of the Combination with AraC

The results shown in Table 5 indicate that on human colon carcinoma HCT-116 cell line the administration of Compound 1 in combination with AraC resulted in a synergistic antitumor effect.

TABLE 5
In vitro evaluation of the cytotoxic effect of Compound 1 in
combination with AraC

| | Drug | | | | |
|---|---|---|---|---|---|
| | Compound 1 | AraC | | | Effect of |
| Cell line | μM | nM | Schedule | C.I. | combination |
| HCT-116 | 0.5 | 0.25 | Sequential | 0.46 | Synergism |
| | 0.25 | 0.25 | | 0.40 | Synergism |

### Example 7

### In Vivo Antitumor Efficacy in Combination with Gemcitabine

Balb, Nu/Nu male mice, from Harlan (Italy), were maintained in cages with paper filter cover, food and bedding sterilized and water acidified. Fragments of BX-PC3 human pancreatic carcinoma were implanted subcutaneously in athymic mice. This tumor model was selected because it was previously demonstrated that it is sensitive to gemcitabine and also because gemcitabine is the standard treatment for pancreatic cancer. The treatment with gemcitabine started at day 9 after tumor implantation, when tumors were palpable. Compounds were prepared immediately before treatments.

Compound 1 of formula (A) was administrated by intraperitoneal route in a volume of 10 ml/kg at the dose of 15 mg/kg twice a day (BID) for 9 consecutive days (days 9, 10, 11, 12, 13, 14, 15, 16 and 17). Gemcitabine was administered intravenously in a volume of 10 ml/kg at the dose of 80 mg/kg for 3 times every 4 days (at days 9, 13, 17). When combined, gemcitabine was administered at days 9, 13, 17 and compound 1 was administered for the following 3 days after gemcitabine administration. Tumor growth and body weight were measured every 3 days. Tumor growth was assessed by caliper. The two diameters were recorded and the tumor weight was calculated according the following formula: length (mm)×width²/2. The effect of the antitumor treatment was evaluated as percentage of tumor growth inhibition (TGI %). Toxicity was evaluated on the basis of body weight reduction. The results are reported in Table 6. Compound 1 combined with the antimetabolite agent gemcitabine produced an additive/synergic effect.

TABLE 6
In vivo efficacy of the combination with gemcitabine

| Treatment | TGI % | Toxicity |
|---|---|---|
| compound 1 | 53 | 0/8 |
| 15 mg/kg* | | |
| gemcitabine 80 mg/kg** | 61 | 0/8 |
| Gemcitabine 80 mg/kg + | 70 | 0/8 |
| compound 1 | | |
| 15 mg/kg*** | | |

*Treatments made intraperitoneally twice at days 9-17
**Treatments made intravenously at days 9, 13, 17
***gemcitabine treatments on day 9, 13, 17; compound 1 treatments on day 10, 11, 12, 14, 15, 16, 18, 19, 20

### Example 8

### In Vivo Antitumor Efficacy in Combination with Cisplatin

Balb, Nu/Nu male mice, from Harlan (Italy), were maintained in cages with paper filter cover, food and bedding sterilized and water acidified. A2780 human ovarian carcinoma cells were obtained from American Type Culture Collection (Rockville, Md.) and were maintained in vitro as continuous cultures in RPMI medium supplemented with 10% FBS, at 37° C., 5% CO₂. For in vivo experiments 8×10⁶ A2780 cells were implanted subcutaneously in athymic mice. This tumor model was selected because it was sensitive to platinum derivatives and also on the basis of use of this drugs in ovarian cancer.

The treatment started when tumors were palpable (day 13). Both compounds were prepared immediately before treatment.

Compound of 1 of formula (A) was administered by intraperitoneal route in a volume of 10 ml/kg at the dose of 15 mg/kg twice a day (BID) from day 13 to day 17. Cisplatin was administered by intravenous route in a volume of 10 ml/kg at the dose of 8 mg/kg at day 5. When combined, compound 1 was administered from day 1 to day 5, cisplatin at day 17. Tumor growth and body weight were measured every 3 days. Tumor growth was assessed by caliper. The two diameters were recorded and the tumor weight was calculated according the following formula: length (mm)×width/2. Animals were sacrificed on day 37 after tumor implant, day 24 from the start of treatments and tumors were weighted. The effect of the antitumor treatment was evaluated as percentage of tumor growth inhibition of the treated group respect to control. Toxicity was evaluated on the basis of body weigh reduction. The results are reported in Table 7.

TABLE 7
In vivo efficacy of the combination with cisplatin

| Treatment | TGI % | Toxicity |
|---|---|---|
| Compound 1 | 46 | 0/7 |
| 15 mg/kg* | | |
| Cisplatin 8 mg/kg | 21 | 0/7 |
| Cisplatin 8 mg/kg + | 77 | 0/7 |
| Compound 1 | | |
| 15 mg/kg*** | | |

*Treatments made ip twice at days 13, 14, 15, 16, 17.
**Treatments made by intravenous route at days 17
***Days 17: cisplatin treatments, days 13-17: Compound 1 treatments

Compound 1 of formula (A) combined with the alkylating agent cisplatin produced a clear synergistic effect. No toxicity was observed in any of the treatment groups.

### Example 9

### In Vivo Antitumor Efficacy of the Combination with Irinotecan

Balb, Nu/Nu male mice, from Harlan (Italy), were maintained in cages with paper filter cover, food and bedding sterilized and water acidified. A2780 human ovarian carcinoma cells were obtained from American Type Culture Collection (Rockville, Md.) and were maintained in vitro as continuous cultures in RPMI medium supplemented with 10% FBS, at 37° C., 5% CO₂. For in vivo experiments 8×10⁶ A2780 were implanted subcutaneously in athymic mice. This tumor model was selected because it was sensitive to platinum derivatives and also on the basis of use of this drugs in ovarian cancer.

The treatment started when tumors were palpable (day 13). Both compounds were prepared immediately before treatment.

Compound 1 of formula (A) was administered by intraperitoneal route in a volume of 10 ml/kg at the dose of 30 mg/kg twice a day (BID) from day 13 to day 17. Irinotecan was administered by intravenous route in a volume of 10 ml/kg at the dose of 60 mg/kg at day 1. When combined, compound 1 was administered from day 1 to day 5, irinotecan at day 1. Tumor growth and body weight were measured every 3 days. Tumor growth was assessed by caliper. The two diameters were recorded and the tumor weight was calculated according the following formula: length (mm)×width/2. Animals were sacrificed on day 37 after tumor implant, day 24 from the start of treatments and tumors were weighted. The effect of the antitumor treatment was evaluated as percentage of tumor growth inhibition of the treated group respect to control. Toxicity was evaluated on the basis of body weigh reduction. The results are reported in Table 8.

TABLE 8
In vivo efficacy of the combination with irinotecan

| Treatment | TGI % | Toxicity |
|---|---|---|
| Compound 1 30 mg/kg* | 72.3 | 0/7 |
| irinotecan 60 mg/kg** | 62 | 0/7 |
| irinotecan 50 mg/kg + | 89.9 | 0/7 |
| Compound 1 30 mg/kg*** | | |

*Treatments made ip twice at days 13, 14, 15, 16, 17.
**Treatments made by intravenous route at days 13
***Days 13: irinotecan treatments, days 13-17: Compound 1 treatments

Compound 1 of formula (A) combined with the topoisomerase I inhibitor irinotecan produced a clear synergistic effect. No toxicity was observed in any of the treatment groups.

### Example 10

### In Vivo Antitumor Efficacy of the Combination with Docetaxel

Balb, Nu\Nu male mice, from Harlan (Italy), were maintained in cages with paper filter cover, food and bedding sterilized and water acidified. 2.5×10⁶ DU145 prostate carcinoma cells (from the American Type Culture Collection) were injected subcutaneously in athymic mice. This tumor model was selected because it was previously demonstrated that docetaxel inhibits growth of the model in vivo (see for reference: Cancer Res. 2004 Oct. 15, (64): 7426-31) and also on the basis of use of this drug in prostate cancer (see, for references, Approval summary: docetaxel in combination with prednisone for the treatment of androgen-independent hormone-refractory prostate cancer, Clin. Cancer Res. 2004 Dec. 15; 10(24): 8147-51).

The treatment started 10 days later tumor cells injection when tumors were palpable. Docetaxel was prepared immediately before treatment, compound 1 of formula (A) was prepared daily, on the basis of known stability of the compound.

Compound 1 was administered by intraperitoneal route in a volume of 10 ml/kg at the doses of 30 mg/kg twice a day (BID) for 9 days (days 10 to 18). Docetaxel was administered by intravenous route in a volume of 10 ml/kg at the dose of 7.5 mg/kg on days 10, 14, 18 from the days of cell injection. When combined, compound 1 was administered in the interval between the docetaxel treatments at days 11, 12, 13, 15, 16, 17, 19, 20 and 21. Tumor growth and body weight were measured every 3 days. Tumor growth was assessed by caliper. The two diameters were recorded and the tumor weight was calculated according the following formula:

length (mm)×width²/2. The effect of the antitumor treatment was evaluated as the delay in the onset of an exponential growth of the tumor (see for references, Anticancer drugs 7:437-60, 1996). This delay (T-C value) was defined as the difference of time (in days) required for the treatment group (T) and the control group (C) tumors to reach a predetermined size (1 g). Toxicity was evaluated on the basis of body weigh reduction. The results were reported in table below. Compound of formula (A) combined with docetaxel produced a clear synergistic effect: The T-C observed when compound 1 was combined with docetaxel was superior (11.5 days) to the expected by the simple addition of T-C (9) obtained by the single treatments. No toxicity was observed in any of the treatment groups. The results are reported in Table 9.

TABLE 9
In vivo efficacy of the combination with docetaxel

| Treatment | T-C (days) | Toxicity |
|---|---|---|
| Compound 1 30 mg/kg* | 5.3 | 0/7 |
| docetaxel 7.5 mg/kg** | 3.7 | 0/7 |
| docetaxel 7.5 mg/kg + | 11.5 | 0/7 |
| Compound 1 30 mg/kg*** | | |

*Treatments made intraperitoneally twice at days 10-18.
**Treatments made by intravenous route at days 10, 14, 18
*** Days 10, 14, 18: docetaxel treatments, days 11, 12, 13, 15, 16, 17, 19, 20 and 21: Compound 1 treatments

1. A therapeutic combination comprising (a) Compound 1 of formula (A): 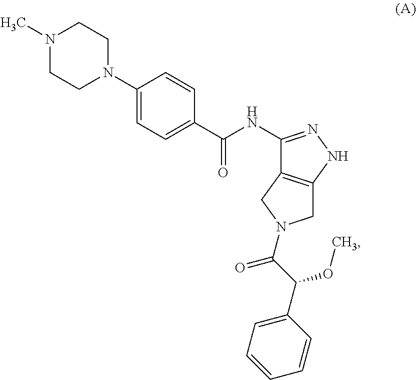
and (b) one or more antineoplastic agents selected from the group consisting of platinum derivatives, antimetabolite agents, topoisomerase I inhibitors and antimicrotubules agents, wherein the active ingredients of the combination are present in each case in free form or in the form of a pharmaceutically acceptable salt or any hydrate thereof.

2. The combination according to claim 1 wherein the platinum derivative is selected from the group consisting of cisplatin, oxaliplatin, carboplatin, nedaplatin, lobaplatin and satraplatin.

3. The combination according to claim 2 wherein the platinum derivative is cisplatin or oxaliplatin.

4. The combination according to claim 1 wherein the antimetabolite agent is selected from the group consisting of 5-fluorouracil (5-FU), capecitabine, gemcitabine, cytosine arabinoside (Ara-C), pemetrexed, methotrexate and edatrexate.

5. The combination according to claim 4 wherein the antimetabolite agent is gemcitabine, 5-FU or Ara-C.

6. The combination according to claim 1 wherein the topoisomerase I inhibitor is selected from the group consisting of topotecan, irinotecan, SN-38 and 9-nitrocamptothecin.

7. The combination according to claim 6 wherein the topoisomerase I inhibitor is irinotecan or SN38.

8. The combination according to claim 1 wherein the antimicrotubule agent is selected from the group consisting of paclitaxel, docetaxel and epothilons derivatives.

9. The combination according to claim 8 wherein the antimicrotubule agent is docetaxel.

10. The combination according to claim 1 which is a combined preparation for simultaneous, separate or sequential use.

11. A method of treating or delaying the progression of a proliferative disorder comprising a simultaneous, sequential or separate administration to a patient in need thereof of a therapeutically effective amount of the combination according to claim 1.

12. A pharmaceutical composition comprising a combination according to claim 1 admixed with a pharmaceutically acceptable carrier, diluent or excipient.

13. A method of treating a proliferative disorder comprising simultaneously, sequentially or separately administering to a patient in need thereof a therapeutically effective amount of the combination according to claim 1 wherein said combination comprising the compound 1 of formula (A) and a platinum derivative, an antimetabolite agent or a topoisomerase I inhibitor.

14. A method for lowering the side effects caused by antineoplastic therapy with an antineoplastic agent in mammals, including humans, in need thereof, the method comprising administering to said mammal a combined preparation comprising compound 1 of formula (A) according to claim 1 and one or more antineoplastic agents selected from the group consisting of platinum derivatives, antimetabolite agents, topoisomerase I inhibitors and antimicrotubule agents, in amounts effective to produce a synergistic antineoplastic effect.

15. A commercial package comprising, in a suitable container means, (a) compound 1 of formula (A) as defined in claim 1, and (b) one or more antineoplastic agents selected from the group consisting of platinum derivatives, antimetabolite agents, topoisomerase I inhibitors and antimicrotubule agents wherein the active ingredients are present in each case in free form or in the form of a pharmaceutically acceptable salt or any hydrate thereof, together with instructions for simultaneous, separate or sequential use thereof.
